(19) United States
(12) Patent Application Publication
(10) Pub. No.: US 2017/0112455 A1
(43) Pub. Date: Apr. 27, 2017

(54) DEFLECTION COMPENSATING MECHANISM FOR MEDICAL IMAGING

(71) Applicant: GENERAL ELECTRIC COMPANY, Schenectady, NY (US)

(72) Inventors: Michael George Bonner, Wauwatosa, WI (US); Adam Clark Nathan, Milwaukee, WI (US)

(21) Appl. No.: 14/918,789

(22) Filed: Oct. 21, 2015

(51) Int. Cl. A61B 6/04; A61G 13/06; A61G 13/04

(52) U.S. Cl. CPC A61B 6/0407; A61G 13/04; A61G 13/06

(57) ABSTRACT

A patient support system is provided. The patient support system includes a support table including a first end and a second end. The patient support system also includes a patient support coupled to the support table and configured to support a subject during an imaging procedure, wherein a top surface of the patient support is angled relative to a bottom surface of the patient support to position a portion of the subject to be imaged to be orthogonal to an imaging scan plane while the patient support is extended from the second end.

### BACKGROUND

The subject matter disclosed herein relates to medical imaging and, in particular, to the use of a mechanism to compensate for deflection or sag of a table or patient support (e.g., a change in table inclination when extended) during medical imaging.

Non-invasive imaging technologies allow images of the internal structures or features of a patient to be obtained without performing an invasive procedure on the patient. In particular, such non-invasive imaging technologies rely on various physical principles, such as the differential transmission of X-rays through the target volume or the emission of gamma radiation, to acquire data and to construct images or otherwise represent the observed internal features of the patient.

Traditionally, medical imaging systems, such as a computed tomography (CT) imaging system or a CT/positron emission tomography (PET) imaging system, include a gantry and a patient table. The patient table needs to be as transparent as possible to X-rays used by the CT imaging system and the gamma rays used by the PET imaging system. As a result, the tables are constructed of thin, composite materials which need to support several hundred pounds of weight. The patient table includes a patient support (e.g., cradle or pallet) that extends from the table into the gantry bore. However, due to the size and weight of the patient and the composition of the table, the vertical position of the patient changes with respect to the imaging gantry due to sagging or deflection of the table and the patient support. For some CT and CT/PET imaging applications, particularly radiation therapy treatment planning, the sagging or deflection may affect the accuracy of localizing a radiation treatment.

### BRIEF DESCRIPTION

In one embodiment, a patient support system is provided. The patient support system includes a support table including a first end and a second end. The patient support system also includes a patient support coupled to the support table and configured to support a subject during an imaging procedure, wherein a top surface of the patient support is angled relative to a bottom surface of the patient support to position a portion of the subject to be imaged to be orthogonal to an imaging scan plane while the patient support is extended from the second end.

In an additional embodiment, a patient support is provided. The patient support includes a radiation therapy table top configured to be disposed on a cradle coupled to a support table having a first end and a second end. The radiation therapy table top includes a top surface configured to interface with a subject to be imaged. The radiation therapy table top includes a bottom surface configured to interface with the cradle. The top surface is non-parallel relative to the bottom surface to enable a portion of the subject to be imaged to be orthogonal to an imaging scan plane while the cradle is extended from the second end for an imaging procedure.

In a further embodiment, a method is provided. The method includes obtaining a weight and a height of a subject to be imaged and an orientation of the subject during an imaging procedure. The method also includes determining an angle of a top surface of a patient support relative to a bottom surface of the patient support, wherein the patient support is coupled to a support table and configured to support the subject during the imaging procedure and to extend away from the support table for the imaging procedure. The method further includes adjusting the angle of the top surface of the patient support. The method yet further includes placing the subject on the patient support and extending the patient support into position for the imaging procedure. The method still further includes performing the imaging procedure on the subject, wherein the top surface of the patient support is angled relative to the bottom surface of the patient support to position a portion of the subject being imaged orthogonal to an imaging scan plane.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 is a combined pictorial view and block diagram of a computed tomography (CT) imaging system as discussed herein;

FIG. 2 depicts a diagrammatic side view of a patient support having an angled top surface versus a patient support having a non-angled top surface, in accordance with aspects of the present description;

FIG. 3 depicts a diagrammatic side view of a patient support having a non-linear top surface, in accordance with aspects of the present description

FIG. 4 depicts a diagrammatic side view of a patient support having an angled top surface with an adjustment mechanism (e.g., shims), in accordance with aspects of the present description;

FIG. 5 depicts a diagrammatic side view of a patient support having an angled top surface with an adjustment mechanism (e.g., screw mechanism), in accordance with aspects of the present description;

FIG. 6 depicts a flow chart of a method for adjusting an angle of a patient support, in accordance with aspects of the present description;

FIG. 7 depicts a diagrammatic side view of a patient support having an angled top surface (e.g., having a cantilevered cradle), in accordance with aspects of the present description;

FIG. 8 depicts a diagrammatic side view of a patient support having an angled top surface (e.g., utilized in conjunction with a tilted table top); and

FIG. 9 depicts a diagrammatic side view of a patient support having an angled top surface (e.g., cradle with angled top surface).

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers'specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As described herein, in certain instances medical imaging systems, such as a computed tomography (CT) imaging system or a CT/positron emission tomography (PET) imaging system, include a patient table that includes a patient support (e.g., cradle or pallet) that extends from the table into a gantry bore. However, due to the size and weight of the patient and the composition of the table, a vertical position of the patient changes with respect to the imaging gantry when the table (e.g., patient support) is extended due to sagging or deflection of the table and the patient support. For some CT and CT/PET imaging applications, particularly radiation therapy treatment planning, the sagging or deflection may affect the accuracy of localizing a radiation treatment. Typical approaches to address this issue include utilizing complex mechanical means to adjust the table and/or patient support to remove the deflection or compensate for it. Alternative approaches include compensating for the deflection or sagging during processing.

In contrast to these approaches, the presently contemplated CT or CT/PET patient support system design introduces a slope in the patient support (e.g., table and/or cradle) to match and compensate for the changing table deflection as the patient travels through a scan plane. The slope of the patient support compensates for the sag downwards as the patient travels through the bore, resulting in a portion of the patient being imaged being flat and level at the imaging plane (i.e., orthogonal to the imaging plane). Embodiments of the presently contemplated patient support system include a top surface of the patient support (e.g., cradle or radiation therapy table top) to be angled relative to a bottom surface or a longitudinal axis of the patient support. Such a configuration enables the portion of the patient being imaged to be at a same height as the scan or imaging plane while the patient support is extended from the table during an imaging operation. In particular CT and CT/PET imaging applications, such as radiation therapy planning, this enables a radiation treatment to be more accurately localized.

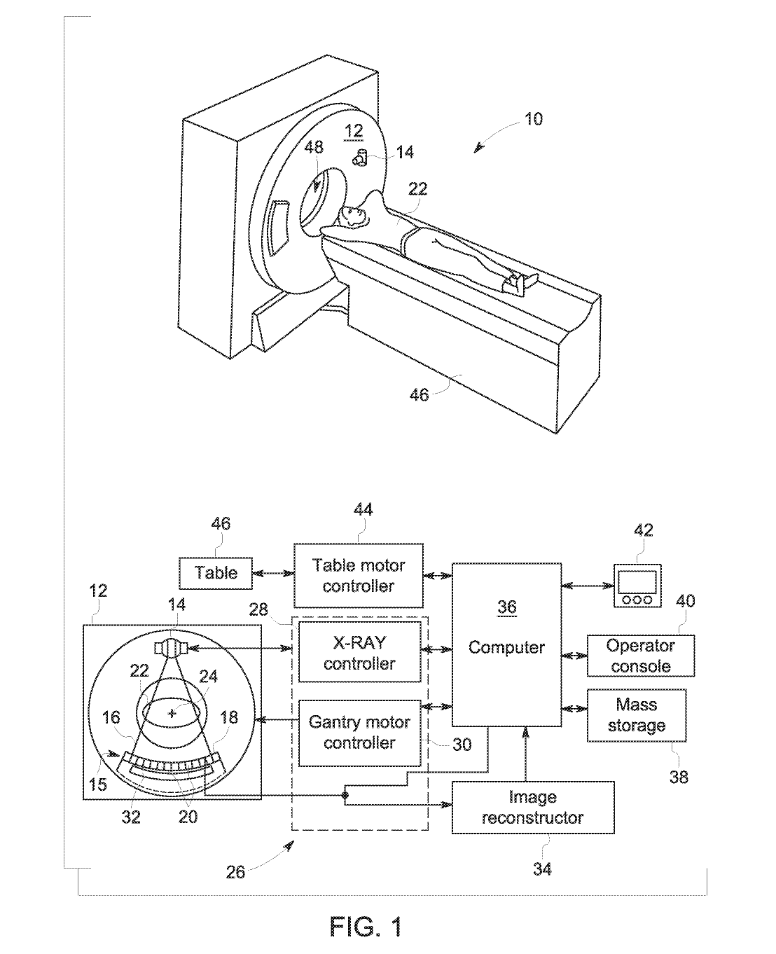

Although the following embodiments are discussed in terms of a computed tomography (CT) imaging system, the embodiments may also be utilized with other imaging systems (e.g., PET, CT/PET, SPECT, nuclear CT, etc.). With the preceding in mind and referring to FIG. 1, a CT imaging system 10 is shown, by way of example. The CT imaging system includes a gantry 12. The gantry 12 has an X-ray source 14 that projects a beam of X-rays 16 toward a detector assembly 15 on the opposite side of the gantry 12. The detector assembly 15 includes a collimator assembly 18, a plurality of detector modules 20, and data acquisition systems (DAS) 32. The plurality of detector modules 20 detect the projected X-rays that pass through a patient 22, and DAS 32 converts the data to digital signals for subsequent processing. Each detector module 20 in a conventional system produces an analog electrical signal that represents the intensity of an incident X-ray beam and hence the attenuated beam as it passes through the patient 22. During a scan to acquire X-ray projection data, gantry 12 and the components mounted thereon rotate about a center of rotation 24 so as to collect attenuation data from a multitude of view angles relative to the imaged volume.

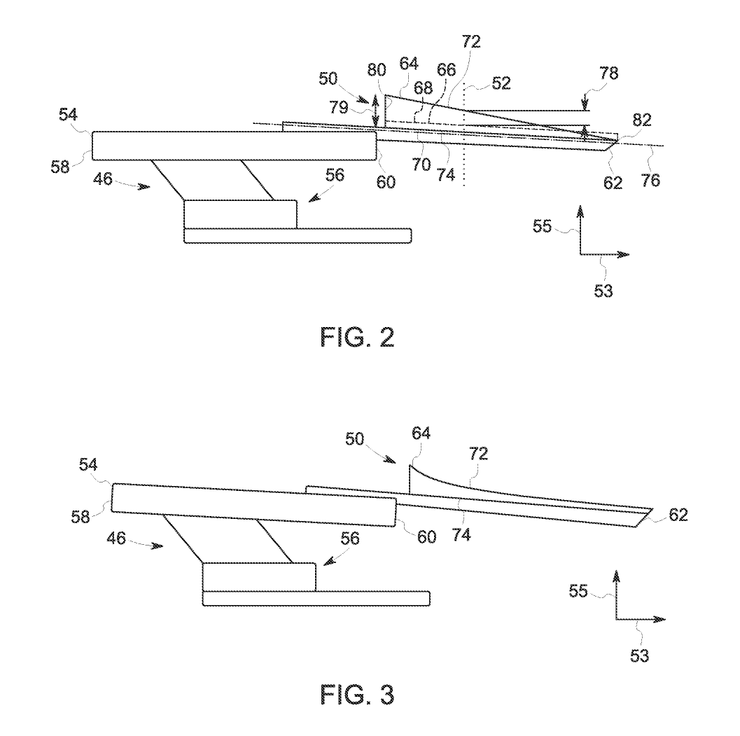

Rotation of gantry 12 and the operation of X-ray source 14 are governed by a control mechanism 26 of CT system 10. Control mechanism 26 includes an X-ray controller 28 that provides power and timing signals to an X-ray source 14 and a gantry motor controller 30 that controls the rotational speed and position of gantry 12. An image reconstructor 34 receives sampled and digitized X-ray data from DAS 32 and performs high-speed reconstruction. The reconstructed image is applied as an input to a computer 36, which stores the image in a mass storage device 38. Computer 36 also receives commands and scanning parameters from an operator via console 40. An associated display 42 allows the operator to observe the reconstructed image and other data from computer 36. The operator supplied commands and parameters are used by computer 36 to provide control signals and information to DAS 32, X-ray controller 28, and gantry motor controller 30. In addition, computer 36 operates a table motor controller 44, which controls a motorized table 46 (and/or patient support such as a cradle and/or radiation therapy table top as shown in FIGS. 2-5 and 7-9) to position patient 22 and gantry 12. Particularly, table 46 moves (e.g., extends) portions of patient 22 on the patient support through a gantry opening or bore 48. As described in greater detail below, the patient support (e.g., cradle and/or radiation therapy table top) includes an angled top surface to compensate for sagging or deflection of the table 46 and/or the patient support as the patient support is extended away from the table toward the gantry opening 48.

FIG. 2 depicts a diagrammatic view of a patient support 50 having an angled top surface versus a patient support 50 having a non-angled top surface. In particular, FIG. 2 depicts how the angled top surface compensates for the sagging or deflection of the table 46 and/or the patient support 50 to enable a portion of the patient to be imaged to be orthogonal relative to a scan plane 52 of the imaging system 10. For illustrative purposes, the gantry 12 is not shown. The table 46 and the patient support 50 may extend along an axial direction 53 and a radial direction 55. The table 46 includes a top portion 54 and a base portion 56. The top portion 54 includes a first end 58 and a second end 60. The top portion 54 is coupled to the patient support 50. The base portion 56 supports the top portion 54 and the patient support 50 (and the patient). In particular, as the patient support 50 is extended away from the table 46 (e.g., from the second end 60) the table 46 supports the patient support 50 (and the patient). The patient support 50 includes a cradle or pallet 62 and a radiation therapy table top 64. The cradle 62 and the radiation therapy table top 64 may be made of X-ray transmissive material (e.g., plastic or carbon fiber) so as to prevent interference with the imaging process (e.g., artifact formation). The cradle 62 includes an arcuate-shaped surface for receiving the patient during typical imaging procedures. However, during radiation therapy planning, the radiation therapy table top 64 provides a surface spanning the arcuate-shaped cradle 62 for the patient support 50 to receive the patient. A typical radiation therapy table top 66 (shown in dashed lines) does not have a top surface 68 that is angled (e.g., non-parallel) relative to a bottom surface 70 of the radiation therapy table top 66. Thus, when the patient support 50 is extended away from the table 46, the patient support 50 (including the radiation therapy table top 66) and table 46 experience sagging or deflection under the weight of the patient (not shown) altering the height of the patient support 50 relative to the imaging scan plane 52. In contrast, the radiation therapy table top 64 includes a top surface 72 (configured to interface with the patient) that is angled (e.g., not parallel) relative to a bottom surface 74 (configured to interface with the cradle 62) or a longitudinal axis 76 of the patient support 50. Thus, when the patient support 50 is extended away from the table 46, the angled top surface 72 of the radiation therapy table top 64 compensates for sagging or deflection (as indicated by reference numeral 78 indicating the difference in height between the table tops 64, 66) under the weight of the patient so that the portion of the patient (not shown) to be imaged is flat and level (i.e., orthogonal) with respect to the imaging scan plane 52. In particular CT and CT/PET imaging applications, such as radiation therapy planning, this enables a radiation treatment to be more accurately localized, when the patient is put on a treatment table.

As depicted, the radiation therapy table top 64 includes a height 79 that decreases from a first end 80 to a second end 82 (point of the patient support 50 furthest away from the second end 60 of the table 46). In certain embodiments, the angle of the top surface 72 of the radiation therapy table top 64 relative to the bottom surface 74 or the longitudinal axis 76 is greater than 0 degree and equal to or less than 5 degrees. In some embodiments, the angle of the top surface 72 of the radiation therapy table top 64 relative to the bottom surface 74 or the longitudinal axis 76 is greater than 0 degree and equal to or less than 1 degree. In certain embodiments, the angle of the top surface 72 is fixed. In other embodiments, the angle of the top surface 72 may be adjusted via an adjustment mechanism (e.g., one or more shims or a screw mechanism). In some embodiments, different radiation therapy table tops 64 with different angles may be utilized. In certain embodiments, the top surface 72 includes a linear shape (e.g., based on a linear function) as depicted in FIG. 2. In other embodiments, the top surface 72 includes a non-linear shape (e.g., based on an n^{th} order curve), as depicted in FIG. 3, to match and compensate for non-linear deflection of the table 46 and the patient support 50.

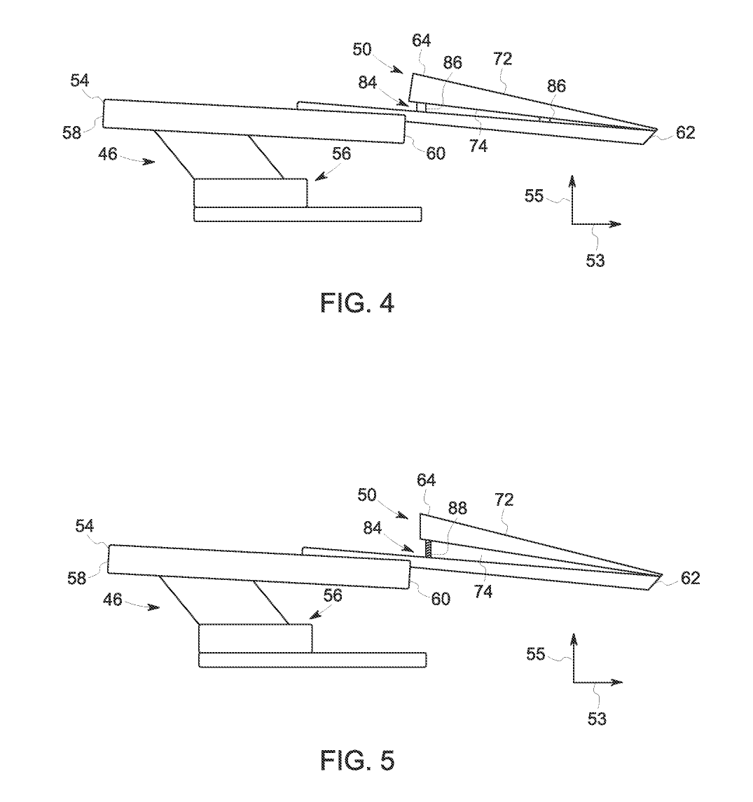

As mentioned above, in certain embodiments, the angle of the top surface 72 of the radiation therapy table tap 64 may be adjusted. The angle of the top surface 72 may be determined based on the height and weight of the patient and/or the orientation of the patient during the imaging procedure (e.g., head-first or feet-first). As depicted in FIGS. 4 and 5, an adjustment mechanism 84 is utilized to adjust the angle of the top surface 72. In FIG. 4, the adjustment mechanism 84 includes utilizing one or more shims 86 to adjust the angle. One or more shims 86 (of the same size or different sizes) may be utilized to adjust the angle of the top surface 72. In FIG. 5, the adjustment mechanism 84 includes a screw mechanism 88 (e.g., threaded bar or rod) to adjust the angle of the top surface 72. The screw mechanism 88 may be manually adjusted or adjusted via an actuator (e.g., in response to control signals from a controller such as controller 44).

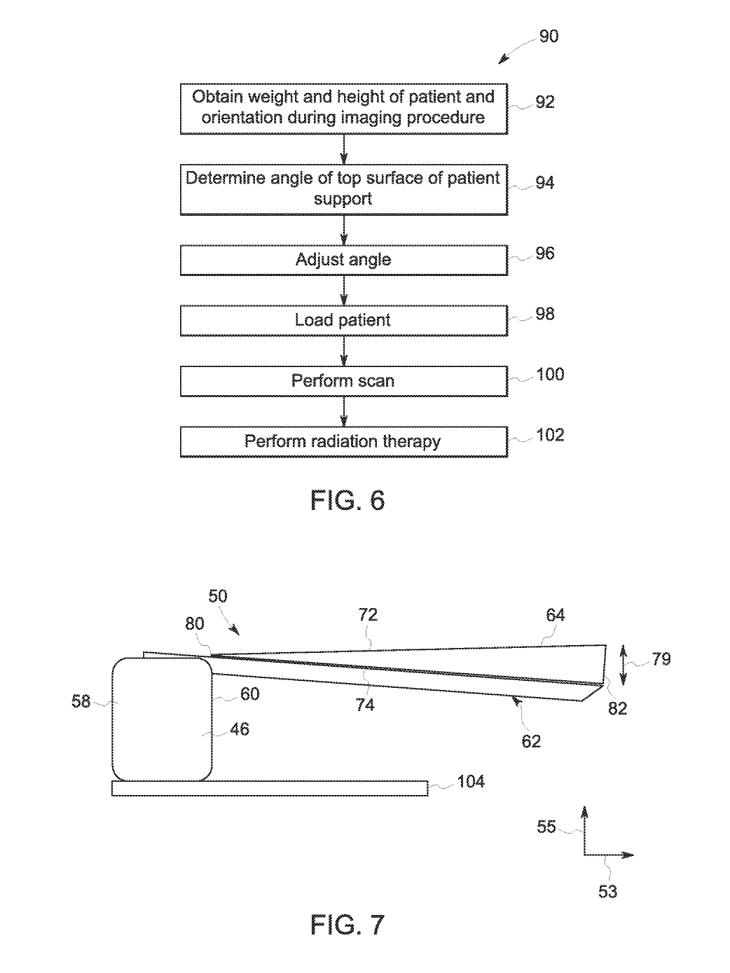

FIG. 6 depicts a flow chart of a method 90 for adjusting an angle of the patient support 50, in accordance with aspects of the present description. The method 90 includes obtaining a weight and a height of the patient and an orientation of the patient during the imaging procedure (e.g. head-first or feet-first) (block 92). The method 90 also includes determining an angle of the top surface of the patient support 50 (e.g., top surface 72 of the radiation therapy table top 64) (block 94). Determining the angle may include utilizing a look-up table (e.g., for a particular table 46 and a particular radiation therapy table top 64) that has a predetermined angle for a given height, weight, and orientation or solving a provided algorithm by providing relevant factors or parameters. The method 90 further includes adjusting the angle of the top surface of the patient support 50 (block 96). The adjustment of the angle of the top surface of the patient support 50 may be conducted via the adjustment mechanism 84 described above. Alternatively, in certain embodiments, a radiation therapy table top 64 having an angled top surface 72 may be selected from a plurality of radiation therapy table tops 64 having different angled top surfaces 72. The method 90 still further includes placing or loading the patient onto the patient support 50 and extending the patient support 50 into position (e.g., within the bore of the gantry 12) for the imaging procedure (block 98). The method 90 yet further includes performing the scan or imagining procedure on the patient where the portion of the patient being imaged is orthogonal to the imaging scan plane 52 (block 100). In certain embodiments, the method 90 still further includes performing the radiation therapy treatment on the patient (block 102). In certain embodiments, the radiation therapy treatment may be performed on a separate table.

FIG. 7 depicts a diagrammatic side view of the patient support 50 having an angled top surface (e.g., having a cantilevered cradle 62). As depicted, the patient support 50 (specifically, the cradle 62) is coupled to the table 46 in a cantilevered arrangement so that the patient support 50 extends in the axial direction 53 away from the second end 60 of the table 46. The cradle 62 is fixed in this position relative to the table 46. In order to move the patient into position into the bore of the gantry 12, the table 46 is coupled to a mobility system 104 (e.g., rail system) that is configured to move both the table 46 and the patient support 50 as one back and forth in the axial direction 53. As depicted, the radiation therapy table top 64 is disposed on top of the cradle 62. As described above, the top surface 72 of the radiation therapy top 64 is angled relative to the bottom surface 74 as described above. However, the radiation therapy table top 64 in FIG. 7 has the opposite orientation than the radiation therapy table top 64 in FIG. 2. In particular, the height 79 of the radiation therapy table top 64 increases from the first end 80 to the second end 82 (point of the patient support 50 furthest away from the second end 60 of the table 46). In certain embodiments, the cantilevered cradle 62 may include an angled top surface to compensate for sagging or deflection and be utilized with a flat radiation therapy table top.

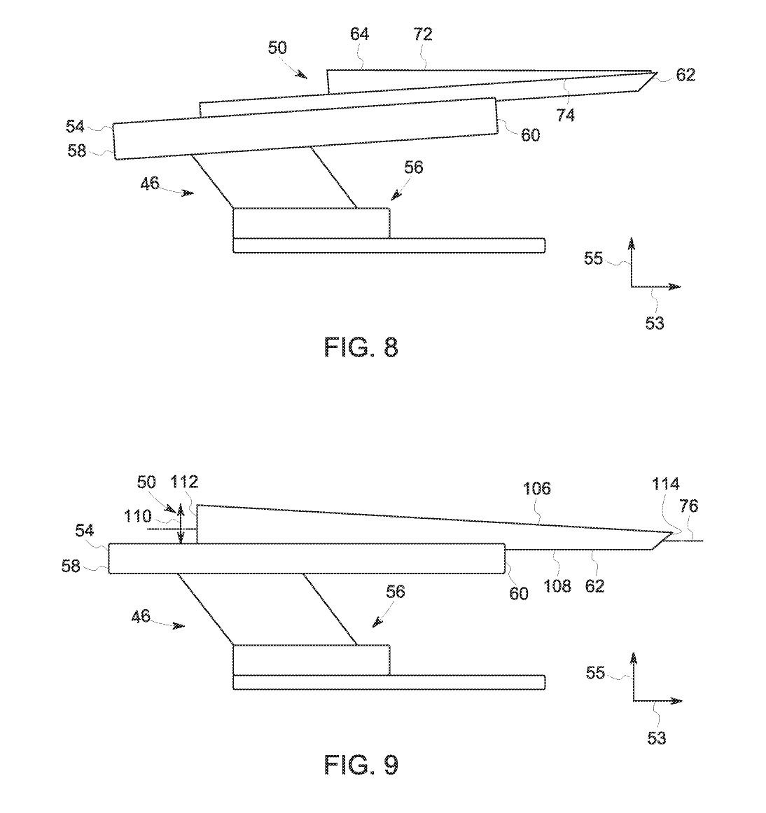

FIG. 8 depicts a diagrammatic side view of the patient support 50 having an angled top surface (e.g., utilized in conjunction with a tilted table 46). As depicted, the table 46 and the patient support 50 are as described above (e.g., in FIG. 2) except the table 46 includes an upward tilt. In particular, the top portion 54 of the table 46 is angled upward (e.g., in the radial direction 55 from the base portion 56 toward the top portion 54) away from the ground or surface the table 46 is disposed on. The angled top portion 54 of the table 46 may assist in addressing differences in heights due to deflection between a room coordinate system and a height of the patient support 50 at the imaging scan plane 52.

FIG. 9 depicts a diagrammatic side view of the patient support 50 having an angled top surface (e.g., the cradle 62 with angled top surface). In certain embodiments, the cradle 62 may include a top surface 106 that is angled (e.g., non-parallel) relative to a bottom surface 108 of the cradle 62 or the longitudinal axis 76 of the patient support 50. As depicted, the cradle 62 includes a height 110 that decreases from a first end 112 to a second end 114 (point of the patient support 50 furthest away from the second end 60 of the table 46). In certain embodiments, the height 110 increases form the first end 112 to the second end 114. In certain embodiments, the angle of the top surface 106 of the cradle 62 relative to the bottom surface 108 or the longitudinal axis 76 is greater than 0 degree and equal to or less than 5 degrees. In some embodiments, the angle of the top surface 106 of the cradle 62 relative to the bottom surface 108 or the longitudinal axis 76 is greater than 0 degree and equal to or less than 1 degree. The angled top surface 106 of the cradle 62 is fixed. In certain embodiments, the top surface 106 includes a linear shape (e.g., based on a linear function) as depicted in FIG. 9. In other embodiments, the top surface 106 includes a non-linear shape (e.g., based on an n^{th} order curve), similar to the top surface 72 of the radiation therapy table top 64 depicted in FIG. 3, to match and compensate for non-linear deflection of the table 46 and the patient support 50. In certain embodiments, the cradle 62 with the angled top surface 106 may be utilized with a flat radiation therapy table top or with a radiation therapy table top with an angled top surface.

Technical effects of the disclosed embodiments include a patient support having an angled top surface. Technical effects also include utilizing the angled top surface of the patient support to enable a portion of the patient to be imaged to be orthogonal to an imaging scan plane during an imaging procedure.

This written description uses examples to disclose the subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A patient support system, comprising:
a support table comprising a first end and a second end; and
a patient support coupled to the support table and configured to support a subject during an imaging procedure and to extend from the second end of the support table for the imaging procedure, wherein a top surface of the patient support is angled relative to a bottom surface of the patient support to position a portion of the subject to be imaged to be orthogonal to an imaging scan plane while the patient support is extended from the second end.

2. The patient support system of claim 1, wherein the patient support comprises a cradle having the top surface.

3. The patient support system of claim 2, wherein an angle of the top surface is fixed.

4. The patient support system of claim 1, wherein the patient support comprises a radiation therapy table top having the top surface.

5. The patient support system of claim 4, wherein the radiation therapy table top comprises a height that decreases from a first end to a second end, and the second end is disposed adjacent a point of the patient support located furthest away from the second end of the support table.

6. The patient support system of claim 4, wherein the radiation therapy table top comprises a height that increases from a first end to a second end, and the second end is disposed adjacent a point of the patient support located furthest away from the second end of the support table.

7. The patient support system of claim 4, wherein the patient support comprises the radiation therapy table top disposed on top of a cradle, and the cradle is cantilevered from the support table.

8. The patient support system of claim 7, comprising a rail system configured to axially move the support table and patient support.

9. The patient support system of claim 4, wherein the support table comprises a top portion coupled to the patient support and a base portion configured to support the top portion, and the top portion is angled upward in a radial direction from the base portion toward the patient support.

10. The patient support system of claim 4, wherein an angle of the top surface is configured to be adjusted.

11. The patient support system of claim 10, comprising an adjustment mechanism configured to adjust the angle of the top surface.

12. The patient support system of claim 11, wherein the adjustment mechanism comprises one or more shims or a screw mechanism.

13. The patient support system of claim 1, wherein an angle of the top surface of the patient support is 5 degrees or less and greater than 0 degree.

14. The patient support system of claim 13, wherein the angle of the top surface of the patient support is 1 degree or less and greater than 0 degree.

15. The patient support system of claim 1, wherein the top surface of the patient support comprises a linear shape.

16. The patient support system of claim 1, wherein the top surface of the patient support comprises a non-linear shape.

17. A patient support, comprising:
a radiation therapy table top configured to be disposed on a cradle coupled to a support table having a first end and a second end, wherein the radiation therapy table top comprises:
a top surface configured to interface with a subject to be imaged; and
a bottom surface configured to interface with the cradle, wherein the top surface is non-parallel relative to the bottom surface to enable a portion of the subject to be imaged to be orthogonal to an imaging scan plane while the cradle is extended from the second end for an imaging procedure.

18. The patient support of claim 17, wherein the top surface of the patient support comprises a linear shape.

19. The patient support of claim 17, wherein the top surface of the patient support comprises a non-linear shape.

20. A method, comprising:
obtaining a weight and a height of a subject to be imaged and an orientation of the subject during an imaging procedure;
determining an angle of a top surface of a patient support relative to a bottom surface of the patient support, wherein the patient support is coupled to a support table and configured to support the subject during the imaging procedure and to extend away from the support table for the imaging procedure;
adjusting the angle of the top surface of the patient support;
placing the subject on the patient support and extending the patient support into position for the imaging procedure; and
performing the imaging procedure on the subject, wherein the top surface of the patient support is angled relative to the bottom surface of the patient support to position a portion of the subject being imaged orthogonal to an imaging scan plane.
